(19) United States
(12) Patent Application Publication
(10) Pub. No.: US 2007/0059391 A1
(43) Pub. Date: Mar. 15, 2007

(54) Antioxidant skin balm

(76) Inventor: Judith Louise Sanderson Perrin, Enville, TN (US)

Correspondence Address: Judith S. Perrin, 2985 Sand Mountain Road, Enville, TN, 38332, US

(21) Appl. No.: 11/513,541

(22) Filed: Sep. 1, 2006

Related U.S. Application Data
(60) Provisional application No. 60/716,754, filed on Sep. 13, 2005.

(51) Int. Cl. A61K 36/73; A61K 36/87

(52) U.S. Cl. 424/765; 424/766

(57) ABSTRACT

This invention pertains to therapeutic antioxidant-lesion/abrasion/mole healing and/or removal compositions comprising therapeutically effective amount of antioxidant/free-radical fighting agents. In one embodiment, the lesion healing composition comprises (a) honey; (b) grape seed extract/proanthocyanidins powder; (c) ascorbic acid (vitamin C with rosehips); (d) d-alpha tocopheryl acetate (natural vitamin E); (e) soy oil; (f) purified water, grape seed oil. The therapeutic antioxidant may be used topically (on external skin) or orally (in mouth or throat areas). The antioxidant composition is sticky in texture and not suitable for applying over a wide area as a moisturizer.

### BACKGROUND OF THE INVENTION

### a) Description

This invention relates to a topical and/or oral compound which has shown capability of exhibiting a removing/restoring/healing ability in human skin abrasions. It relates to antioxidant/free-radical fighting to provide positive results to skin abrasions. Skin abrasions include damage to surface tissues due to irritations, such as crusty lesions, basil cell carcinoma, melanoma, harm caused by the sun, inflammation of a mole or a raised skin area.

General treatment of these skin abrasions is to apply as stated below and then to await spontaneous recovery due to the antioxidant/free-radical fighting ability of its agents. Treatment may take affect over a period of a few days or as long as a month for desired results.

The method of treating a lesion/abrasion/mole topically consists of applying the composition to the affected area of the skin daily, covering with a bandage strip, and not cleansing the affected area between applications. The method of treating a lesion in the mouth or throat consist of applying the composition directly to the affected area as needed. The invention applies to skin ailments. Ingestion of large amounts to treat a so-called internal ailment is not recommended.

Honey acts as an antibiotic agent while the other agents in the composition of the antioxidant skin balm serve to fight abnormal appearances on the skin, possibly caused by free radical invasion. Combined, these agents in the composition of the antioxidant skin balm have shown success in eliminating and repairing various skin damage. The antioxidant skin balm composition is sticky in texture and not suitable for massaging over a wide area as a moisturizer.

### b) DISCLOSURE OF THE INVENTION AND EXAMPLES OF USE

The present inventor, after studying the effects of free-radical damage and agents to fight this damage, invented the said antioxidant skin balm when a large, black crust lesion appearing on her husband's ear was diagnosed by a family physician and a surgeon as cancerous. Upon using the antioxidant skin balm on her husband's ear, the lesion was healed completely and no surgery or other treatment was necessary.* As others have used the antioxidant skin balm they, too, have had successful results.*
*See enclosed literature

8. An object of the present invention is therefore to provide an antioxidant/free-radical fighting composition, which exhibits good spreadability and compatibility on the skin lesion/abrasion/mole, and permits easy application to the damaged skin area.

No particular limitations of use of the present invention have been set, other than not to use the antioxidant skin balm near the eyes, inside the ear or nose as the acids in its composition could cause a burning effect. No pain has been reported by users of this invention to the inventor. After having hundreds of users of this invention no complaint from any user has been received by the inventor.

Users of this antioxidant skin balm are urged to follow the instructions for use diligently, which are as stated: Apply balm to the affected area of the skin daily, cover with a bandage strip. For best results do not wash balm off of the affected area before reapplying each day. Cover well and keep off clothes, etc. to guard against staining. Store at room temperature in a cool, dry place. Avoid excessive heat. Do not refrigerate. Use within 6 months. In natural ingredients consistency may vary. If thickening occurs the balm may be thinned with pure (preferably raw) honey.

Although it has not yet been advertised to outside customers, the inventor has seen positive results when using the antioxidant balm on skin abrasions and/or soreness in the mouth or throat simply by applying directly, as needed, on the affected area, therefore she is including this use as part of patent application.

This antioxidant invention contains no preservatives since its compensation is comprised of only natural ingredients, yet the honey appears to act as a preserving agent and the invention's composition has shown to have a shelf life of approximately 1½ years. For best results it should always be stored at room temperature. The antioxidant invention is made by simply mixing the composition agents until desired consistency is obtained.

### Precise Composition of Antioxident Skin Balm

20-28 ounces of honey, depending on the thickness of the honey
3 cups of herbal grape seed/proanthocyanindin extract standardized powder
200,000 mg. of Vitamin C
40,000 mg. of rose hips
20,000 IU d-alpha tocopheryl acetate (Vitamin E) in a base of soy oil, glycerin and purified water
½ teaspoon (or trace amount) of grape seed oil
   I declare the above statements to be true as far as my knowledge and I declare that I am the sole inventor of this Antioxidant Skin Balm. Judith S. Perrin

I claim:

1. A therapeutic composition pertaining to antioxidant-tissue healing/restoring agents useful for reducing the size, duration, and severity of lesions, abrasions and moles. Adapted for oral and non-oral application in humans comprising an effective amount of a mixture of honey and agents high in natural antioxidants: grape seed extract/proanthocyanindin powder, vitamin C with rose hips, natural vitamin E comprised in soy oil and purified water, and grape seed oil.

2. The method of treating a lesion/abrasion/mole wherein the surface tissue consists of skin, administered by applying antioxidant skin balm invention to a person in need thereof a composition comprising antioxidant/free-radical fighting agents for healing.

3. The method of treating a lesion/abrasion/mole topically consists of applying the composition to the affected area of the skin daily and covering with a bandage strip and not cleansing the affected skin area between applications.

4. The method of treating a lesion in the mouth or throat consists of applying the composition directly to the affected area as needed.

5. Benefit of prior filed co-pending provisional application 60/716,754

6. No related or similar product is known by the inventor
